(19) United States
(12) Patent Application Publication
(10) Pub. No.: US 2014/0343159 A1
(43) Pub. Date: Nov. 20, 2014

(54) FORMULATION OF SMALL ADRENERGIC AGONIST SALT FORMS IN ORGANIC SOLVENTS

(71) Applicant: PROCERTUS BIOPHARM, INC., Madison, WI (US)

(72) Inventor: William E. Fahl, Fitchburg, WI (US)

(73) Assignee: ProCertus Biopharm, Inc., Madison, WI (US)

(21) Appl. No.: 14/122,442

(22) PCT Filed: Mar. 9, 2012
(86) PCT No.: PCT/US2012/028446

§ 371 Date: Mar. 18, 2014

Related U.S. Application Data
(60) Provisional application No. 61/492,664, filed on Jun. 2, 2011.

(51) Int. Cl. A61K 47/10; A61K 31/138

(52) U.S. Cl. CPC A61K 47/10; A61K 31/138
USPC 514/653

(57) ABSTRACT

Aspects of the present disclosure are directed to formulations that permit the solubilization and long term, stable storage of adrenergic agonist vasoconstrictors in organic solvents. Provided herein are formulations comprising about 250 mM to about 3000 mM of an acid salt of an adrenergic agonist vasoconstrictor dissolved in a pharmaceutically acceptable topical delivery vehicle comprising a water miscible, organic solvent having a water content of less than 40% by weight. Also provide are methods for using and kits containing such formulations.

### CROSS REFERENCE TO RELATED APPLICATIONS

This application claims priority to U.S. Provisional App. No. 61/492,664, filed Jun. 2, 2011, the entire contents of which are incorporated herein by reference.

### TECHNICAL FIELD

The present invention relates to the field of drug formulation. In particular, the invention provides methods for the solubilization and long term, stable storage of vasoconstrictors in organic solvents.

### BACKGROUND

The use of chemotherapy and radiotherapy to treat cancer patients is associated with several severe side-effects. These treatments are toxic to highly-mitotic, epithelial stem cell populations within the hair follicle and epidermis and often result in side effects such as alopecia and radiation dermatitis. Because there are currently no treatments to prevent these cancer therapy side-effects the full utility of chemotherapeutic drugs and radiation therapy (also referred to herein as “radiotherapy”) in the treatment of cancer has not been fully exploited.

The application of vasoconstrictors has been shown to prevent radiation-induced dermatitis in a rat model when applied topically to the radiation site prior to irradiation (see U.S. Pub. No 2007/0077219, filed Jun. 19, 2006). In the model, a topical vasoconstrictor was applied to a shaved area on the backs of rats prior to irradiation with a Cs¹³⁷ source. The severity of radiodermatitis was scored on day 13. Vasoconstriction at the topical treatment site was evidenced by the transient occurrence of a visible skin blanch that correlates with therapeutic efficacy in the rat radiodermatitis model. This skin blanch may serve as a surrogate efficacy marker for clinical studies. The general strategy of using topical vasoconstrictors to prevent these cancer therapy side effects is addressed in U.S. Pub. No 2007/0077219, which is incorporated by reference in its entirety herein.

It is generally understood that topical delivery of a drug into and through the skin (“transdermal delivery”) requires that the majority of the topical delivery vehicle be an organic solvent (Tsai, J.-C., Weiner, N. D., Flynn, G. L., and Ferry, J. J. Drug and Vehicle Deposition From Topical Applications: Localization of Minoxidil within Skin Strata of the Hairless Mouse. Skin Pharmacol., 7: 262-269, 1994). The use of organic solvents, however, does have drawbacks. For example, if high concentrations of the vasoconstrictor are required for treatment, these higher concentrations of vasoconstrictor may be insoluble in the organic solvent. A vasoconstrictor formulation with the ability to prevent radiation induced dermatitis and alopecia without the drawback of chemical insolubility at high concentrations would be highly desirable.

### SUMMARY

The present disclosure pertains to the discovery that, inter alia, an acid salt of an adrenergic agonist vasoconstrictor molecule can dissolve and be stably stored as a product both: (i) in a >60% solvent vehicle, and (ii) at very high concentrations (for example, greater than 250 mM), both of which are preferred for delivery of such compounds through skin.

In one aspect, the present application provides formulations for topical delivery of a vasoconstrictor to a subject comprising about 250 mM to about 3000 mM of an acid salt of an adrenergic agonist vasoconstrictor dissolved in a pharmaceutically acceptable topical delivery vehicle comprising a water miscible, organic solvent having a water content of less than 40% by weight.

Also disclosed are methods comprising applying to a subject's skin a formulation comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight.

In another aspect, the present application provides kits comprising a container housing an aliquot of a formulation for topical delivery of a vasoconstrictor comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight; and, an applicator for applying the aliquot to a subject's skin.

Other features of the present disclosure are described herein.

### BRIEF DESCRIPTION OF THE DRAWINGS

The foregoing and other aspects of the present invention will become apparent from the following detailed description of the invention when considered in conjunction with the accompanying drawing. For the purpose of illustrating the invention, there is shown in the drawing an embodiment that is presently preferred, it being understood, however, that the invention is not limited to the specific instrumentality disclosed.

FIG. 1 shows photographs of the backs of three radiation-treated rats, showing the occurrence of severe radiodermatitis in one rat treated with delivery vehicle alone (control, Rat A) and the absence of radiodermatitis in two rats treated with 600 mM (−) norepinephrine-HCl dissolved in an ethanol:water (70:30) delivery vehicle (Rats B,C) prior to irradiation.

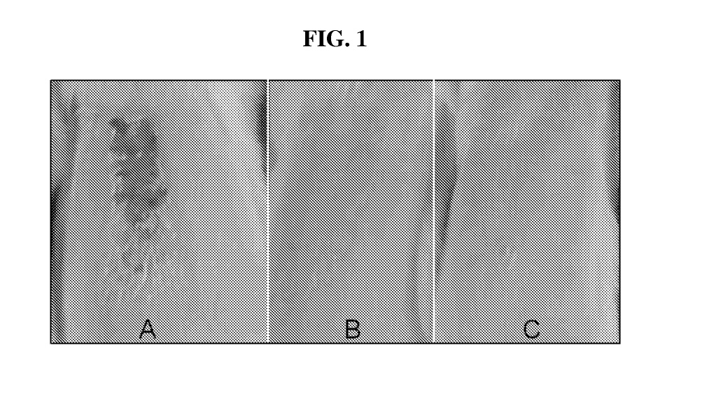

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

In the present disclosure the singular forms “a,” “an,” and “the” include the plural reference, and reference to a particular numerical value includes at least that particular value, unless the context clearly indicates otherwise. Thus, for example, a reference to “a solvent” may be a reference to one or more of such solvents and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Unless otherwise specified, any component, element, attribute, or step that is disclosed with respect to one embodiment of the present methods and products may apply to any other method or product that is disclosed herein.

The disclosures of each patent, patent application, and publication cited or described in this document are hereby incorporated herein by reference, in their entirety.

The practical utility of previous vasoconstrictor formulations has been restricted by limitations on the concentration of drug that could be dissolved within the delivery vehicle. The solubility limit for norepinephrine tartrate in suitable delivery vehicles is about 200 mM. However, successful treatment, for example, prophylactic application of vasoconstrictor to an area that may be vulnerable to radiation or chemotherapy induced toxicity, may require a higher concentration of norepinephrine, which has traditionally been incompatible with preferred delivery vehicles. The present inventors have surprisingly discovered that the acid salt of adrenergic agonist vasoconstrictors can be dissolved at considerably higher concentrations in optimal delivery vehicles as may be required for efficacious treatment (whether prophylactic or otherwise) of conditions such as radiation dermatitis, alopecia, mucositis, proctitis, and gastrointestinal distress.

Accordingly, provided herein are formulations for topical delivery of a vasoconstrictor to a subject comprising about 250 mM to about 3000 mM of an acid salt of an adrenergic agonist vasoconstrictor dissolved in a pharmaceutically acceptable topical delivery vehicle comprising a water miscible, organic solvent having a water content of less than 40% by weight.

Suitable vasoconstrictors for use in the present formulations include agonists of the α₁ adrenergic receptor. Exemplary agonists of the α₁ adrenergic receptor include epinephrine, phenylephrine, methoxamine, norepinephrine, tetrahydrozaline, naphazoline, or any combination thereof. In certain embodiments, the vasoconstrictor is a racemic (+/−) mixture of the acid salt a given vasoconstrictor, for example, norepinephrine. In other embodiments, the vasoconstrictor is a single enantiomer of the acid salt, such as, for example, (−) norepinephrine. The vasoconstrictor salt form may be (−) norepinephrine-HCl, (−) norepinephrine-HBr, (−) norepinephrine-HF, or (−) norepinephrine-HI. Preferably, the vasoconstrictor has suitable solubility characteristics in high percentage organic solutions preferred for topical, transdermal drug delivery.

Topical epinephrine applied prior to irradiation of rat skin was shown to confer complete protection against subsequent development of radiation dermatitis. Shortly after this, topically applied norepinephrine was shown to confer the same protection. Because norepinephrine does not bind to the β2 adrenergic receptor, it does not elicit some of the cardiac side effects associated with epinephrine, such as tachycardia, arrhythmia, etc. At least for this reason, as well as its potency in binding the α1 adrenergic receptor that induces vasoconstriction, norepinephrine is well-suited for use as the active agent for a topical, vasoconstrictor drug for the prevention of radiodermatitis. Traditionally, a super-saturated solutions of (−) norepinephrine-tartrate, which is stable for only a few hours, has been used as a vasoconstrictor for topical application for prevention of the dermatitis and alopecia seen in rodents exposed to either or both radiation and chemotherapy. The solubility of (−) norepinephrine-tartrate in organic solvents, however, is limited.

Without being limited to any particular theory of operation, it is postulated that vasoconstrictor acid salts having a lower formula weight have increased solubility in organic solvents, compared to traditional vasoconstrictors. For example, (−) norepinephrine-tartrate (FW: 337) salt is traditionally used in vasoconstrictor formulations and its maximum solubility in a typical ethanol and water solution at a ratio of 70:30, by volume is about 200 mM. The present inventors, however, have discovered that lower formula weight salts of vasoconstrictors have much higher solubility in organic solutions. Lower molecular weight vasoconstrictor salts include, for example, a halide acid salt. For example, the vasoconstrictor may be used in the form of a hydrochloride salt (HCl), a hydrobromide salt (HBr), a hydrofluoride salt (HF), a hydroiodide salt (HI), or any combination thereof. The vasoconstrictor may also or alternatively be an acetate salt or an hydrogen sulfate (H₂SO₄) salt.

The present vasoconstrictor acid salts may be dissolved in the pharmaceutically acceptable topical delivery vehicle at a concentration (and therefore have a solubility in the vehicle) of about 250 mM to about 3000 mM, for example, about 400 mM to about 2000 mM, or, stated differently, about 250 mM, about 300 mM, about 400 mM, about 500 mM, about 600 mM, about 700 mM, about 800 mM, about 900 mM, about 1000 mM, about 1200 mM, about 1400 mM, about 1600 mM, about 1800 mM, about 2000 mM, about 2200 mM, about 2400 mM, about 2600 mM, about 2800 mM, or about 3000 mM. As used herein, a vasoconstrictor is “dissolved” in the delivery vehicle when the resulting formulation contains substantially no undissolved vasoconstrictor after having been stored at room temperature (e.g., about 63° F. to 74° F.) for at least 60 days, a characteristic that has not been previously achievable at a vasoconstrictor concentration of greater than about 200 mM, as described more fully infra.

Suitable water miscible, organic solvents for use in the delivery vehicle include, for example, ethanol, propanol, isopropanol, acetone, propylene glycol, glycerol, polyethylene glycol, butanediol, dimethyl sulfoxide (DMSO), or a combination thereof. The organic solvent is ideally biocompatible, can dissolve the vasoconstrictor, and is capable of delivering the vasoconstrictor transdermally. For example, preferable solvents may be particularly good at delivering the vasoconstrictor to dermal vasculature that underlies skin or may allow penetration of stratum corneum and hair follicle sebum residue. Additionally, preferred water miscible, organic solvents may provide for fast drying on the skin and ease of analysis of the vasoconstrictor after formulation.

The delivery vehicle may include a water miscible, organic solvent having a water content of less than about 40% by weight. Thus, in certain embodiments, the vasoconstrictor may be dissolved in a solvent comprising non-aqueous and aqueous components. The delivery vehicle may comprise alcohol and water. For example, the delivery vehicle may comprise alcohol and water in a ratio of about 60-80 to about 20-40 (i.e., 60-80:20-40).

The vasoconstrictor of the present formulations may be stabilized through the inclusion of an antioxidant. Suitable antioxidants include citric acid, ethylenediaminetetraacetic acid (EDTA), metabisulfite, ascorbic acid, butylated hydroxytoluene (BHT), butylated hydroxyanisole (BHA), or a combination thereof. The antioxidant may be present in an amount of from about 0.005 to about 0.5 percent weight of the solution. The addition of the antioxidant may provide for solution stability for at least about 90 days, 180 days, or 365 days at room temperature. Preferably the solution is stable for at least one year, more preferably, at least 2 years. For example, the solution may be stable for about 1 year, about 2 years, about 3 years, about 4 years, about 5 years, about 1 year to about 2 years, about 1 year to about 3 years, about 2 years to about 3 years, about 3 years to about 4 years, or about 3 years to about 5 years.

Also provided herein are methods comprising applying to a subject's skin a formulation comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight.

The characteristics of the formulation that is applied to a subject's skin pursuant to the present methods may correspond to those that are described in connection with the presently disclosed formulations, supra.

The formulations may be applied to the subject's skin using any suitable process for application of topical delivery vehicle. For example, the formulation may be applied manually, using an applicator, or by a process that involves both. Following application, the formulation may be worked into the subject's skin, e.g., by rubbing. Application may be performed multiple times daily or on a once-daily basis. For example, the formulation may be applied to a subject's skin once a day, twice a day, or multiple times a day, or may be applied once every two days, once every three days, or about once every week, once every two weeks, or once every several weeks, depending on the degree of risk of radiation or chemotherapy induced toxicity to the subject or of actual radiation or chemotherapy induced toxicity.

The formulation may be applied in a volume of about 0.1 mL to about 5.0 mL per 25 cm² of the subject's skin. For example, with respect to the specified area of skin, the formulation may be applied in a volume of about 0.1 mL to about 4.0 mL, about 0.5 mL to about 3.0 mL, or about 0.5 mL to about 2.0 mL. The total volume applied per session of application may depend on the concentration of vasoconstrictor in the formulation; the actual dosage may be determined according to standard dosing calculations.

In another aspect, the present application provides kits comprising a container housing an aliquot of a formulation for topical delivery of a vasoconstrictor comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight, and an applicator for applying the aliquot the subject's skin. The characteristics of the formulation that is included in the present kits may correspond to those that are described in connection with the presently disclosed formulations, supra.

An aliquot may comprise about 0.1 mL to about 100 mL of the formulation. It may be of use to the practitioner for a given aliquot to correspond to the amount of formulation that should be applied to a given area of the subject's skin, such that during application, when the container that houses the aliquot is empty, the practitioner knows not to apply any additional formulation to that area of skin. For example, if the designated area of skin has an area of about 25 cm², then a container may house an aliquot having a volume of about 0.1 mL to about 4.0 mL, about 0.5 mL to about 3.0 mL, or about 0.5 mL to about 2.0 mL.

The kit may include a single container or may include multiple containers. When the kit includes multiple containers, all of the containers may respectively house the same volume of formulation, or at least two of the containers may respectively house different volumes of the formulation. For example, the kit may include a further container that houses a volume of formulation that is greater than the aliquot in the original container. In some instances, the kit may comprise a plurality of containers, wherein at least one container houses a volume of formulation that is greater than the aliquot, and/or at least one other container houses a volume of formulation that is less than the aliquot. Depending on the risk of radiation or chemotherapy induced toxicity or of actual radiation or chemotherapy induced toxicity with respect to a particular area of skin, a larger or smaller volume of formulation may be required. For example, given a kit that comprises a plurality of containers, the practitioner may choose a container from the kit that contains a larger volume of formulation when the objective is to apply that volume to an area of skin that is at greater risk of or actually suffers from radiation or chemotherapy induced toxicity. On the other hand, given the same kit, the practitioner may choose a container from the kit that contains a smaller volume of formulation when the risk of or actual toxicity to a different area of skin is comparatively low.

The size and configuration of the applicator may vary depending upon the needs of the user. The applicator may comprise, for example, a sponge-like material. In certain embodiments, particularly for the application of formulation to a scalp, the applicator may be in the form of a comb with hollow teeth, such that the formulation may be combed through the hair and applied directly to the scalp. In another embodiment, e.g., for the purpose of preventing radiotherapy or chemotherapy induced alopecia, the applicator may be in the form of a “swim cap,” where the edge of the cap is a tight band that prevents vasoconstrictor “runoff” from the scalp, and the interior surface of the cap is, for example, a layer of foam that may be wetted with the formulation, which upon placement and sealing of the cap around the scalp hair, the patient could recurrently massage to deposit drug formulation to the scalp prior to and/or during radiotherapy/chemotherapy. The present applicator embraces any configuration that is suitable for topically applying the vasoconstrictor formulation to a subject's skin.

The kit may include multiple applicators having various sizes and shapes. The kit may permit the end user to choose a specific applicator for a particular use. The need for containers of different volumes and applicators of different configurations and/or sizes is driven in significant part by the need to apply this drug to skin sites of widely different area based upon the size of the radiotherapy irradiation field. Field sizes can vary from a few square centimeters on a person's forehead to hundreds of square centimeters over a person's breast, and adjacent axilla, and shoulder.

The present invention is further defined in the following Examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

### Maximum Solubility of (−) Norepinephrine-Tartrate (FW: 337) in a Skin Topical Delivery Formulation of Ethanol:Water (70:30) by Volume

Known weights of (−) norepinephrine-tartrate dry powder were added to individual, 2.0 ml clear glass vials. An appropriate volume of ethanol and water at a ratio of 70:30 by volume solvent was then added to achieve the indicated concentrations. Sealed vials were vortexed for five seconds and then left at room temperature for 24 hr in the dark before examination to determine turbidity of liquid due to undissolved material. Table 1, below, shows the maximum solubility of (−) norepinephrine-tartrate (FW: 337) in topical delivery vehicle of ethanol:water (70:30).

TABLE 1

| Concentration (mM) | Clarity of Solution* |
|---|---|
| 100 | Clear |
| 150 | Clear |
| 200 | Clear |
| 300 | Turbid |
| 400 | Turbid |

*After 24 hr at room temperature

### Example 2

### Solubility of Racemic (+/−) Norepinephrine-HCl (FW: 205) Formulation in Ethanol:Water (70:30)

Whereas (−) norepinephrine-tartrate is maximally soluble at 200 mM concentration in a skin topical delivery formulation of ethanol and water at a ratio of 70:30, by volume, racemic (+/−) norepinephrine-HCl is soluble at much higher concentrations in the same 70:30 solvent.

### Method

Known weights of (−) norepinephrine-tartrate or (+/−) norepinephrine-HCl dry powders were added to individual, 2.0 ml, clear glass vials. An appropriate volume of ethanol to water (70:30) solvent was then added to achieve the indicated concentrations. Sealed vials were vortexed for five seconds and then left at room temperature for 24 hr in the dark before examination to determine turbidity of liquid due to undissolved material. Table 2, below, provides the solubility of (+/−) norepinephrine-HCl and (−) norepinephrine-tartrate in topical delivery vehicle of ethanol and water (70:30).

TABLE 2

| | | Clarity of Solution* |
|---|---|---|
| Molecule | Concentration (mM) | Ethanol:Water (70:30) |
| (−) Norepinephrine-tartrate | 100 | Clear |
| (FW = 337) | 150 | Clear |
| | 200 | Clear |
| | 300 | Turbid |
| | 400 | Turbid |
| (+/−)Norepiniphrine-HCl | 100 | Clear |
| (FW: 205) | 200 | Clear |
| | 300 | Clear |
| | 400 | Clear |
| | 600 | Clear |
| | 800 | Clear |
| | 1000 | Clear |
| | 2000 | Clear |

The solubility of the (+/−) norepinephrine-HCl salt was at least 10-fold better than that seen for (−) norepinephrine-tartrate in the same ethanol and water (70:30) solvent mixture, extending to at least 2000 mM.

### Example 3

### Activity of (+/−) Norepinephrine-HCl

The vasoconstrictor activity of a solution of 600 mM (+/−) norepinephrine-HCl (containing 300 mM of the (−) norepinephrine-HCl enantiomer) in 70:30 ethanol in water delivery vehicle was determined to have at least the same activity in inducing a skin blanch response in human skin following topical application as does a supersaturated solution of 300 mM (−) norepinephrine-tartrate in the 70:30 delivery vehicle.

Appropriate weights of each dry norepinephrine salt were weighed into clear glass vials, and appropriate volumes of ethanol and water at a ratio of 70:30, by volume, were added to achieve the indicated norepinephrine concentrations. The (+/−) norepinephrine-HCl dissolved to clarity with 5 second mixing at room temperature, and the (−) norepinephrine-tartrate required heating to 60° C. for 5 minutes and then mixing to clarify. 25 μL aliquots of each formulation were applied using a pipettor over multiple 2×2 cm patches of skin on volunteer's arm over a 20 second period. Images to record skin blanch onset, degree and duration were begun at 2 minute post-application. % Skin Blanch scores were assigned by visual evaluation. 100% Skin Blanch equaled the skin blanch seen after pressing firmly on the skin and quickly releasing. Table 3, below, shows the induced blanch response in human skin following topical application of 600 mM (+/−) norepinephrine-HCl or 300 mM (−) norepinephrine-tartrate in topical delivery vehicle of ethanol and water (70:30).

TABLE 3

| Norepinephrine | Time After Application to | % |
|---|---|---|
| Formulation | Skin (minutes) | Skin Blanch |
| 600 mM (+/−) | | — |
| Norepinephrine-HCl | 0 min | 0 |
| | 7 min | 10 |
| | 20 min | 40 |
| | 30 min | 80 |
| | 45 min | 85 |
| 300 mM (−) Norepinephrine- | | — |
| tartrate | 0 min | 0 |
| | 7 min | <5 |
| | 20 min | 25 |
| | 30 min | 65 |
| | 45 min | 70 |

Equal concentrations (300 mM) of the (−) norepinephrine enantiomer, either alone as the tartrate salt or in the presence of an equal amount of the (+) enantiomer HCl salt, were applied to human skin in the ethanol:water (70:30) delivery vehicle, and the pharmacologic efficacy of the (−) norepinephrine was scored based upon its ability to induce a skin blanch response by binding to al adrenergic receptors in the smooth muscle cells of the subcutaneous dermal blood vessels. Many published studies have shown that the L (−) enantiomer of catecholamines binds with high affinity to α and β adrenergic receptors, and that the R (+) enantiomers bind with much lower affinity. It is apparent that the (−) norepinephrine enantiomer HCl salt was delivered efficiently to the al adrenergic receptors in the dermal vasculature beneath the human skin, and delivery appeared to be more efficient than for (−) norepinephrine-tartrate, possibly due to the substantial differences in formula weight and hydrophobicity for the two salt forms (i.e., FW: 205 for (−) norepinephrine-HCl vs. FW: 337 for (−) norepinephrine-tartrate).

### Example 4

### Solubility of (−) Norepinephrine-Hcl in Ethanol:Water (70:30)

Whereas (−) norepinephrine-tartrate is maximally soluble at 200 mM concentration in the skin topical delivery formulation of ethanol:water (70:30), (−) norepinephrine-HCl is soluble to at least 2000 mM in the same 70:30 solution.

### Method

Known weights of the indicated norepinephrine salts were added to individual, 2.0 ml, clear glass vials. An appropriate volume of 70:30 (ethanol:water) solvent was then added to achieve the indicated concentrations. Sealed vials were vortexed for five seconds and then left at room temperature for 24 hr before examination to determine turbidity of liquid due to undissolved material. Table 4, below, shows the results of a study designed to assess the maximum solubility of (−) norepinephrine-HCl and (−) norepinephrine-tartrate in topical delivery vehicle of ethanol:water (70:30).

TABLE 4

| Molecule | Concentration (mM) | Clarity of Solution |
|---|---|---|
| (−) Norepinephrine-tartrate | 100 | Clear |
| | 150 | Clear |
| | 200 | Clear |
| | 300 | Turbid |
| | 400 | Turbid |
| (−) Norepinephrine-HCl | 100 | Clear |
| | 200 | Clear |
| | 300 | Clear |
| | 400 | Clear |
| | 600 | Clear |
| | 800 | Clear |
| | 1000 | Clear |
| | 2000 | Clear |

### Example 5

### Stability of (−) Norepinephrine-HCl Dissolved in Water at 1000 mM

(−) norepinephrine-HCl dissolved in water at 1000 mM is stable at 4° C. and 24° C. as determined by the absence of precipitate or colored oxidation products in the weeks/months following initial formulation. Previous formulations of norepinephrine-tartrate in saline (e.g., Levophed®, Hospira, Inc., Lake Forest, Ill.) have norepinephrine concentrations of 1-5 mM.

### Method

(−) norepinephrine-HCl was weighed into a glass vial. An appropriate volume of N₂-purged water (>10 mM with vigorous N₂ bubbling and stir bar) was delivered to the vial. The vial headspace was flushed with N₂ gas and the vial was sealed. The vial was vortexed for 5 seconds to produce a clear solution. A weighed amount of dry citric acid solid was added to the (−) norepinephrine-HCl solution, the vial headspace was flushed with N₂ and the vial was sealed. The vial was vortexed until the solution was clear. 200 μl aliquots of (−) norepinephrine-HCl+citric acid solution were added to 2.0 ml, clear, glass vials (previously rinsed with ethanol and dried). The vial headspaces were optionally flushed with N₂ gas and the vials were sealed. 3.0 ml of (−) norepinephrine-HCl+citric acid solution was transferred to a glass vial, an appropriate dry weight of sodium EDTA was added to the vial, the vial headspace was flushed with N₂ gas, the vial was sealed and the vial was vortexed until clear. 200 μl aliquots of the (−) norepinephrine-HCl+citric acid+EDTA formulation were delivered to 2.0 ml, clear, glass vials. The vial headspaces were optionally flushed with N₂ gas and the vials were sealed. Table 5, below, shows the results of an assessment of the chemical stability of (−) norepinephrine-HCl at high concentration (1000 mM) in water with or without chemical stabilizing strategies.

TABLE 5

| | 1000 mM | | | | | | | | | | | | |
|---|---|---|---|---|---|---|---|---|---|---|---|---|---|
| | (−) NEp- | | | | | | | | | | | | |
| | HCl in N₂- | +0.5% | | N₂ Flush | | | Vials Remaining with No Precipitate or Color | | | | | | |
| Test | purged | Citric | +0.05% | Vial | 4° C. | 24° C. | | | Day | Day | Day | Day | Day |
| Group | water | Acid | EDTA | Headspace | Dark | Dark | Day 0 | Day 5 | 10 | 15 | 31 | 50 | 83 |
| 1 | X | X | X | X | X | | 4 | 4 | 4 | 4 | 4 | 4 | 4 |
| 2 | X | X | X | X | | X | 4 | 4 | 4 | 4 | 4 | 4 | 4 |
| 3 | X | X | X | — | | X | 4 | 4 | 4 | 4 | 4 | 4 | 4 |
| 4 | X | X | — | X | | X | 4 | 4 | 4 | 4 | 4 | 4 | 4 |
| 5 | X | X | — | — | | X | 4 | 4 | 4 | 4 | 4 | 4 | 4 |
| 6 | X | X | — | X | X | | 4 | 4 | 4 | 4 | 4 | 4 | 4 |

### Example 6

### Stability of (−) Norepinephrine-HCl Dissolved in Ethanol:Water (70:30)

### Method

(−) norepinephrine-HCl was weighed into a glass vial. The appropriate volume of N₂-purged 70:30 (ethanol:water; solvents N₂ purged separately then mixed, >10 mM with vigorous bubbling and stir bar) was delivered to the vial. The vial headspace was flushed with N₂ and the vial was sealed. The vial was vortexed for 10 seconds to produce a clear solution. Weighed amounts of dry sodium metabisulfite were added to the NEp-HCl solution (0.2%˜=max. solubility); the vial headspace was flushed with N₂, the vials were sealed and vortexed until clear (˜45 seconds). Using a pipet, 200 ul aliquots of (−) norepinephrine HCl+metabisulfite solution were delivered to 2.0 ml glass vials (previously rinsed with ethanol and dried). The vial headspace was flushed with N₂ (or not) and sealed. 3.0 ml of (−) norepinephrine HCl+metabisulfite solution was transferred to a glass vial. The appropriate dry weight of citrate and/or sodium EDTA was added, the vial headspace was flushed with N₂, the vials were sealed and vortexed until clear (seconds). Using a pipet, 200 ul aliquots of the formulation were added to 2.0 ml glass vials. The vial headspace was flushed with N₂ (or not) and sealed. Each test group was evaluated in triplicate. Table 6, below, shows the results of an assessment of the chemical stability of (−) norepinephrine-HCl at 600 mM in ethanol/water (70/30) with chemical stabilizing strategies at 4° C.

TABLE 6

| | 600 mM | | | | | | | | | | | | |
|---|---|---|---|---|---|---|---|---|---|---|---|---|---|
| | (−) NEp- | 0.2% | | | N₂ | Vials Remaining with No Precipitate or Color | | | | | | | |
| Test | HCl in N₂ | meta | 0.1% | 0.01% | Flush | | | | | Day | Day | Day | Day |
| Group | 70:30 | bisulfite | citrate | EDTA | Vial | Day 0 | Day 1 | Day 2 | Day 6 | 12 | 19 | 38 | 71 |
| 1A | X (not N₂ | | | | 3 | 3 | 3 | 3 | 3 | 3 | 3 | 3 | 3 |
| | 70:30) | | | | | | | | | | | | |
| 1 | X | X | | | 3 | 3 | 3 | 3 | 3 | 3 | 3 | 3 | 3 |
| 2 | X | X | X | | 3 | 3 | 3 | 3 | 3 | 3 | 3 | 3 | 3 |
| 3 | X | X | X | X | 3 | 3 | 3 | 3 | 3 | 3 | 3 | 3 | 3 |
| 4 | X | | | | 3 | 3 | | 3 | 3 | 3 | 3 | 3 | 3 |

Table 7, below, provides the results of a further study concerning the chemical stability of (−) norepinephrine-HCl at 600 mM in ethanol/water (70/30), which demonstrated that solubility of the vasoconstrictor was maintained for at least 178 days.

TABLE 7

| | | | | | | | Vial Incubation | | | | |
|---|---|---|---|---|---|---|---|---|---|---|---|
| | | | | | | | Condition | | | | |
| | Addition to Vial^{c} | | | | | | 40° C. | 24° C. | 4° C. | Vial Appearances | |
| Grp | A | B | C | D | E | F | Dark | Dark | Dark | Day 0^{b} | Day 178 |
| 1 | + | | | | | + | 3^{a} | | | Clear | 40° C.: no ppt; black color |
| 2 | + | + | | | | + | 3 | 3 | 3 | Clear | 40° C.: no ppt; dark brown color |
| | | | | | | | | | | | 24° C.: no ppt; clear and no color |
| | | | | | | | | | | | 4° C.: no ppt; clear and no color |
| 3 | + | + | + | | | + | 3 | 3 | 3 | Clear | 40° C.: no ppt; dark brown color |
| | | | | | | | | | | | 24° C.: no ppt; clear and no color |
| | | | | | | | | | | | 4° C.: no ppt; clear and no color |
| 4 | + | + | | + | | + | 3 | 3 | 3 | Clear | 40° C.: no ppt; dark brown color |
| | | | | | | | | | | | 24° C.: no ppt; clear and no color |
| | | | | | | | | | | | 4° C.: no ppt; clear and no color |
| 5 | + | + | | | + | + | 3 | 3 | 3 | Clear | 40° C.: no ppt; dark brown color |
| | | | | | | | | | | | 24° C.: no ppt; clear and no color |
| | | | | | | | | | | | 4° C.: no ppt; clear and no color |

^{a}Number of vials in indicated incubation setting
^{b}All vials were clear (no color) and had no precipitate
^{c}Addition to Vial:
A. 600 mM L(−) Norepinephrine HCl in N₂-flushed 70:30
B. 0.2% sodium metabisulfite
C. 0.1% sodium citrate
D. 0.2% sodium citrate
E. 0.3% sodium citrate
F. N₂ flush Vial Headspace

Yet another study, the results of which are shown in Table 8, below, demonstrated that the solubility of the vasoconstrictor was maintained for 270 days.

TABLE 8

| | | | | | | Vial Incubation | | | | |
|---|---|---|---|---|---|---|---|---|---|---|
| | | | | | | Condition | | | | |
| | Addition to Vial^{c} | | | | | 4° C. | 24° C. | 40° C. | Vial Appearances | |
| Grp | A | B | C | D | E | Dark | Dark | Dark | Day 0^{b} | Day 270 |
| 1A | +^{d} | | | | | 3^{a} | 3 | 3 | Clear | 40° C.: no ppt; brown color |
| | | | | | | | | | | 24° C.: no ppt; brown color |
| | | | | | | | | | | 4° C.: no ppt; rose color |
| 1 | + | | | | + | 3 | 3 | 3 | Clear | 40° C.: no ppt; brown color |
| | | | | | | | | | | 24° C.: no ppt; brown color |
| | | | | | | | | | | 4° C.: no ppt; rose color |
| 2 | + | + | | | + | 3 | 3 | 3 | Clear | 40° C.: no ppt; dark brown color |
| | | | | | | | | | | 24° C.: no ppt; clear and no color |
| | | | | | | | | | | 4° C.: no ppt; clear and no color |
| 3 | + | + | + | | + | 3 | 3 | 3 | Clear | 40° C.: no ppt; dark brown color |
| | | | | | | | | | | 24° C.: no ppt; clear and no color |
| | | | | | | | | | | 4° C.: no ppt; clear and no color |
| 4 | + | + | + | + | + | 3 | 3 | 3 | Clear | 40° C.: no ppt; dark brown color |
| | | | | | | | | | | 24° C.: needles^{e}; no color |
| | | | | | | | | | | 4° C.: needles; no color |

^{a}Number of vials in indicated incubation setting
^{b}All vials were clear (no color) and had no precipitate
^{c}Addition to Vial:
^{d}Not made with N₂-flushed 70:30
^{e}“Needle” precipitate was EDTA
A. 600 mM L(−) Norepinephrine HCl in N₂-flushed 70:30
B. 0.2% sodium metabisulfite
C. 0.1% sodium citrate
D. 0.01% sodium EDTA
E. N₂ flush Vial Headspace

Table 9, below, shows the results of an assessment of the chemical stability of (−) norepinephrine-HCl at 600 mM in ethanol/water (70/30) with chemical stabilizing strategies at 24° C.

TABLE 9

| | 600 mM | | | | | | | | | | | | |
|---|---|---|---|---|---|---|---|---|---|---|---|---|---|
| | (−) NEp- | 0.2% | | | N₂ | Vials Remaining with No Precipitate or Color | | | | | | | |
| Test | HCl in N₂ | meta | 0.1% | 0.01% | Flush | | | | | Day | Day | Day | Day |
| Group | 70:30 | bisulfite | citrate | EDTA | Vial | Day 0 | Day 1 | Day 2 | Day 6 | 12 | 19 | 38 | 71 |
| 1A | X (not N₂ | | | | 3 | 3 | 3 | 3 | 3 | 3 | 3 | 3 | 3 |
| | 70:30) | | | | | | | | | | | | |
| 1 | X | X | | | 3 | 3 | 3 | 3 | 3 | 3 | 3 | 3 | 3 |
| 2 | X | X | X | | 3 | 3 | 3 | 3 | 3 | 3 | 3 | 3 | 3 |
| 3 | X | X | X | X | 3 | 3 | 3 | 3 | 3 | 3 | 3 | 3 | 3 |
| 4 | X | | | | 3 | 3 | | 3 | 3 | 3 | 3 | 3 | 3 |

Table 10, below, shows the results of an assessment of the chemical stability of (−) norepinephrine-HCl at 600 mM in ethanol/water (70/30) with chemical stabilizing strategies at 40° C.

TABLE 10

| | 600 mM | | | | | | | | | | | | |
|---|---|---|---|---|---|---|---|---|---|---|---|---|---|
| | (−) NEp- | 0.2% | | | N₂ | Vials Remaining with No Precipitate or Color | | | | | | | |
| Test | HCl in N₂ | meta | 0.1% | 0.01% | Flush | | | | | Day | Day | Day | Day |
| Group | 70:30 | bisulfite | citrate | EDTA | Vial | Day 0 | Day 1 | Day 2 | Day 6 | 12 | 19 | 38 | 71 |
| 1A | X (not N₂ | | | | 3 | 3 | 3 | 3 | 3 | 3 | 3 | 3 | 3 |
| | 70:30) | | | | | | | | | | | | |
| 1 | X | X | | | 3 | 3 | 3 | 3 | 3 | 3 | 3 | 3 | 3 |
| 2 | X | X | X | | 3 | 3 | 3 | 3 | 3 | 3 | 3 | 3 | 3 |
| 3 | X | X | X | X | 3 | 3 | 3 | 3 | 3 | 3 | 3 | | 3 |
| 4 | X | | | | 3 | 3 | | 3 | 3 | 3 | 3 | 3 | 3 |

What is claimed:

1. A formulation for topical delivery of a vasoconstrictor to a subject comprising:
about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight.

2. The formulation according to claim 1 wherein said adrenergic agonist vasoconstrictor is epinephrine, norepinephrine, phenylephrine, methoxamine, zolmitriptan, tetrahydrozaline, naphazoline, or any combination thereof.

3. The formulation according to claim 1 wherein the organic solvent is ethanol, propanol, isopropanol, acetone, propylene glycol, glycerol, polyethylene glycol, butanediol, dimethyl sulfoxide, or a combination thereof.

4. The formulation according to claim 1 wherein said delivery vehicle comprises alcohol and water in a ratio of 60-80:20-40.

5. The formulation according to claim 1 comprising a halide acid salt of said vasoconstrictor.

6. The formulation according to claim 5 comprising a hydrofluoride, hydrochloride, hydrobromide, or hydroiodide salt of said vasoconstrictor.

7. The formulation according to claim 1 comprising an acetate or hydrogen sulfate salt of said vasoconstrictor.

8. The formulation according to claim 1 comprising a single enantiomer of said acid salt of said vasoconstrictor.

9. The formulation according to claim 1 comprising a racemic mixture of the (+) and (−) enantiomers of said acid salt of said vasoconstrictor.

10. The formulation according to claim 9 comprising racemic (+/−) norepinephrine acid salt.

11. The formulation according to claim 1 further comprising an antioxidant.

12. The formulation according to claim 11 wherein said antioxidant is citric acid, ethylenediaminetetraacetic acid, metabisulfite, ascorbic acid, butylated hydroxytoluene, butylated hydroxyanisole, or a combination thereof.

13. A method comprising applying to a subject's skin a formulation comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight.

14. The method according to claim 13 wherein said formulation is applied to said subject's skin on a once-daily basis.

15. The method according to claim 13 wherein said formulation is applied in a volume of about 0.5 mL to about 2.0 mL per 25 cm² of said subject's skin.

16. The method according to claim 13 wherein said skin is at risk from radiotherapy- or chemotherapy-induced toxicity.

17. A kit comprising:
a container housing an aliquot of a formulation for topical delivery of a vasoconstrictor comprising about 250 mM to about 3000 mM of an acid salt of an adrenergic agonist vasoconstrictor dissolved in a pharmaceutically acceptable topical delivery vehicle comprising a water miscible, organic solvent having a water content of less than about 40% by weight;
and,
an applicator for applying said aliquot to a subject's skin.
